(19) United States
(12) Patent Application Publication
(10) Pub. No.: US 2018/0140856 A1
(43) Pub. Date: May 24, 2018

(54) SYSTEMS AND METHODS FOR IMPLANTABLE AUTOMATIC MRI MODE ENABLING

(71) Applicant: PACESETTER, INC., Sylmar, CA (US)

(72) Inventors: Brad Lindevig, Santa Monica, CA (US); Frank Wei, Palo Alto, CA (US); Gabriel A. Mouchawar, Valencia, CA (US); Shiloh Sison, Alameda, CA (US); Richard Williamson, Santa Monica, CA (US)

(21) Appl. No.: 15/356,371

(22) Filed: Nov. 18, 2016

(51) Int. Cl. A61N 1/39; A61B 5/055; A61N 1/372

(52) U.S. Cl. CPC A61N 1/3931; A61N 1/37264; A61B 5/055

(57) ABSTRACT

The present disclosure provides systems and methods for an active implantable medical device (AIMD). The AIMD includes a processor, a first magnetic field sensor communicatively coupled to the processor and configured to detect magnetic fields generated by a handheld magnet, and at least one second magnetic field sensor communicatively coupled to the processor and configured to detect magnetic fields generated by a magnetic resonance imaging (MRI) scanner. The processor is configured to sample the first magnetic field sensor and the at least one second magnetic field sensor to detect the presence of the MRI scanner, and automatically initiate an MRI mode for the AIMD based on the detection.

### A. FIELD OF THE DISCLOSURE

The present disclosure relates generally to implanted medical devices, and in particular to implanted medical devices having an automatically enabled and disabled MRI mode.

### B. BACKGROUND ART

Patients implanted with active implantable medical devices (AIMDs) may be at risk when undergoing a magnetic resonance imaging (MRI) scan due to interactions of superconducting, radio frequency (RF), and gradient magnetic fields from the MRI system with the AIMD. However, patients implanted with AIMDs may need an MRI scan as part of their treatment.

AIMD MR Conditional labelling is used to indicate that an AIMD is safe for use within an MRI environment under specified conditions of use for the AIMD therapy, etc. For at least some known AIMDs, those conditions require physicians and MRI technicians to follow detailed technical instructions to manually enable a MRI mode that facilitates preventing potential hazards during an MRI procedure. However, manually programming AIMDs into an MRI mode is inconvenient and may create opportunities for human error in clinical settings.

### BRIEF SUMMARY OF THE DISCLOSURE

In one embodiment, the present disclosure is directed to an active implantable medical device (AIMD). The AIMD includes a processor, a first magnetic field sensor communicatively coupled to the processor and configured to detect magnetic fields generated by a handheld magnet, and at least one second magnetic field sensor communicatively coupled to the processor and configured to detect magnetic fields generated by a magnetic resonance imaging (MRI) scanner. The processor is configured to sample the first magnetic field sensor and the at least one second magnetic field sensor to detect the presence of the MRI scanner, and automatically initiate an MRI mode for the AIMD based on the detection.

In another embodiment, the present disclosure is directed to an automatic magnetic resonance imaging (MRI) mode module for use in an active implantable medical device (AIMD). The automatic MRI mode module includes a processor, a first magnetic field sensor communicatively coupled to the processor and configured to detect magnetic fields generated by a handheld magnet, and at least one second magnetic field sensor communicatively coupled to the processor and configured to detect magnetic fields generated by a magnetic resonance imaging (MRI) scanner. The processor is configured to sample the first magnetic field sensor and the at least one second magnetic field sensor to detect the presence of the MRI scanner, and automatically initiate an MRI mode for the AIMD based on the detection.

In another embodiment, the present disclosure is directed to a method for automatically initiating a magnetic resonance imaging (MRI) mode for an active implantable medical device (AIMD). The method includes sampling, using a processor, a first magnetic field sensor and at least one second magnetic field sensor, wherein the first magnetic field sensor is configured to detect magnetic fields generated by a handheld magnet, and wherein the at least one second magnetic field sensor is configured to detect magnetic fields generated by an MRI scanner. The method further includes detecting the presence of the MRI scanner based on the sampling, and automatically initiating the MRI mode based on the detection.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIGS. 1A and 1B are views of one embodiment of an active implantable medical device (AIMD).

FIG. 2 is a schematic block diagram of an automatic MRI mode module 200 that may be implemented within the AIMD shown in FIG. 1.

FIG. 3 is a schematic diagram of one embodiment of a horizontal Hall sensor that may be used with the automatic MRI mode module shown in FIG. 2.

FIG. 4 is a schematic diagram of one embodiment of a vertical Hall sensor that may be used with the automatic MRI mode module shown in FIG. 2.

FIG. 5 is a flow diagram illustrating one embodiment of a process of a patient implanted with the AIMD shown in FIG. 1 undergoing an MRI scan.

FIGS. 6A-6C are a flow diagram illustrating one embodiment of a method for detecting an MRI scanner.

Corresponding reference characters indicate corresponding parts throughout the several views of the drawings.

### DETAILED DESCRIPTION OF THE DISCLOSURE

The present disclosure provides AIMDs that are capable of automatically detecting an MRI environment, and automatically initiating an MRI mode in response to the detection. This benefits physicians and patients by reducing the inconvenience of an MRI scan, and reducing the probability of error associated with manually activating and deactivating an MRI mode. It also reduces the amount of time the AIMD is in the MRI mode as compared to at least some known MRI mode programming schemes. In the MRI mode, one or more functionalities of the AIMD (e.g., pacing functionality, pacing rate, bipolar vs. unipolar pacing, tachycardia therapy, sensing, input impedance, etc.) are altered or disabled, as will be appreciated by those of skill in the art. The embodiments described herein automatically detect the superconducting magnetic field of an MRI scanner, and then initiate an MRI mode of the AIMD in response to that detection. Once the patient exits the MRI scanner, and the superconducting magnetic field is no longer detected, or after a set duration of time has passed, the AIMD automatically returns to the previous settings (e.g., optimal therapy pacing settings). This also reduces the number of visits that a patient must make to a physician to program the AIMD.

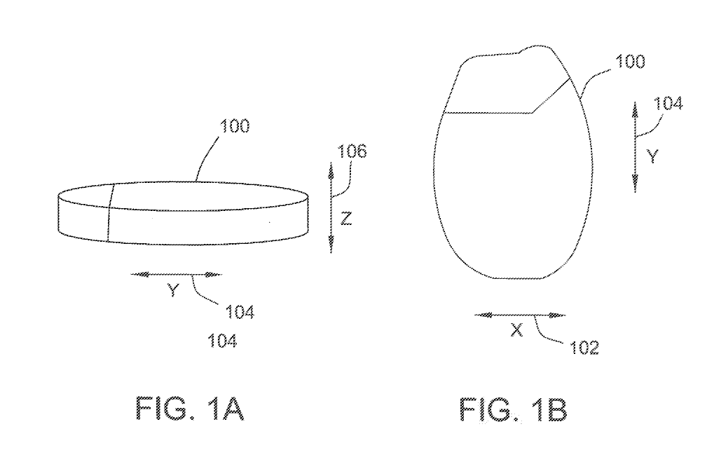

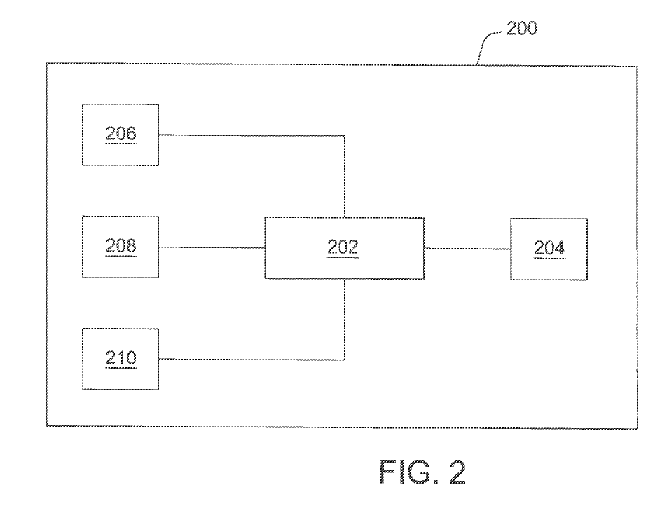

Referring now to the drawings, and in particular to FIGS. 1A and 1B, an active implantable medical device (AIMD) is indicated generally at 100. Specifically, FIG. 1 is a side perspective view of AIMD 100, and FIG. 2 is a front view of AIMD 100. As shown in FIG. 1, AIMD 100 includes three axes: an x-axis 102, a y-axis 104 perpendicular to x-axis 102. and a z-axis 106 perpendicular to both x-axis 102 and y-axis 104. AIMD 100 may be, for example, a pacemaker, a cardiac resynchronization therapy defibrillator (CRT-D), an insertable cardiac monitor (ICM), a deep brain stimulation (DBS) device, a dorsal root ganglia (DRG) stimulation device, a cardiac resynchronization therapy pacer (CRT-P), or a leadless cardiac pacemaker (LCP). Alternatively, AIMD 100 may be any implantable medical device capable of functioning as described herein.

FIG. 2 is a schematic block diagram of an automatic MRI mode module 200 that may be implemented within AIMD 100 (shown in FIG. 1). Automatic MRI mode module 200 includes a processor 202 communicatively coupled to a memory device 204. Processor 202 is also communicatively coupled to a horizontal Hall sensor 206, a first vertical Hall sensor 208, and a second vertical Hall sensor 210. Although Hall sensors are described in this embodiment, those of skill in the art will appreciate that any suitable magnetic field sensor (e.g., a magneto resistor sensor, etc.) may be used in the systems and methods described herein. Hall sensors 206, 208, and 210 facilitate detecting that AIMD 100 is within an MRI environment, and automatically activating an MRI mode for AIMD 100 in response to that detection, as described herein. Processor 202 may include any suitable filtering and/or signal processing circuitry for processing signals received from Hall sensors 206, 208, and 210.

In some embodiments, executable instructions are stored in memory device 204. In the illustrated embodiment, automatic MRI mode module 200 performs one or more operations described herein by programming processor 202. For example, processor 202 may be programmed by encoding an operation as one or more executable instructions and by providing the executable instructions in memory device 204.

Processor 202 may include one or more processing units (e.g., in a multi-core configuration). Further, processor 202 may be implemented using one or more heterogeneous processor systems in which a main processor is present with secondary processors on a single chip. In another illustrative example, processor 202 may be a symmetric multi-processor system containing multiple processors of the same type. Further, processor 202 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In this embodiment, memory device 204 is one or more devices that enable information such as executable instructions and/or other data to be stored and retrieved. Memory device 204 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 204 may be configured to store, without limitation, application source code, application object code, source code portions of interest, object code portions of interest, configuration data, execution events and/or any other type of data.

Each Hall sensor 206, 208, and 210 is a bipolar magnetic field Hall sensor that detects a magnetic field along one of axes 102, 104, and 106. In this embodiment, horizontal Hall sensor 206 detects a magnetic field along z-axis 106, first vertical Hall sensor 208 detects a magnetic field along x-axis 102, and second vertical hall sensor 210 detects a magnetic field along y-axis 104. Processor 202 activates an MRI mode based on the magnetic field detected by Hall sensors 206, 208, and 210, as described herein. In the MRI mode, one or more functionalities of AIMD 100 (e.g., pacing functionality) are altered or disabled, as will be appreciated by those of skill in the art. In an alternative embodiment, automatic MRI mode module 200 includes a single vertical Hall sensor instead of two vertical Hall sensors.

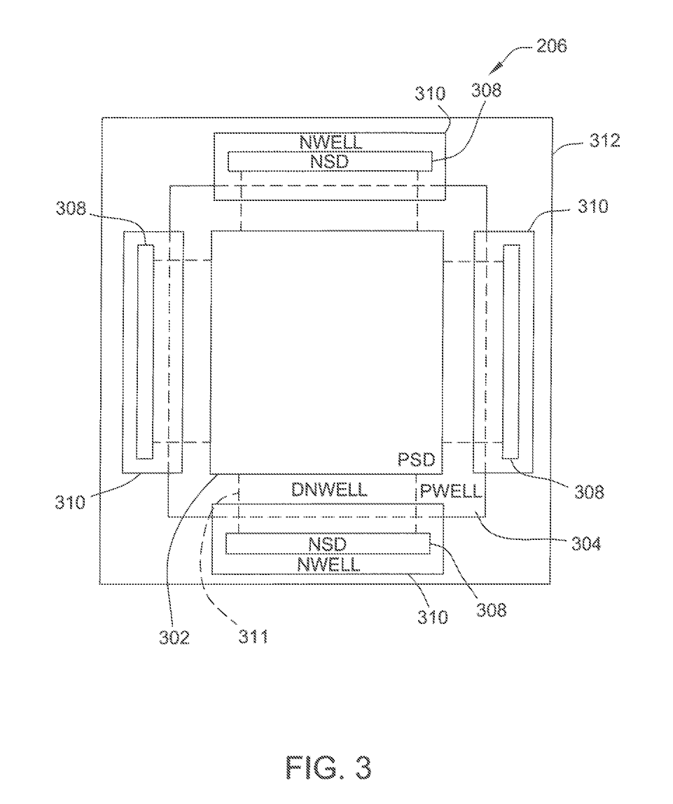

FIG. 3 is a schematic diagram of one embodiment of horizontal Hall sensor 206. Sensor 206 includes a first p-type source drain (PSD) 302 in a p-type well (PWELL) 304. Sensor 206 also includes a plurality of n-type source drains (NSD) 308 in respective n-type wells (NWELL) 310. A deep n-type well (DNWELL) 311 is positioned below PWELL 304, and a second PSD 312 functions as a guarding. Those of skill in the art will appreciate that other sensor architectures may be used to implement horizontal Hall sensor 206.

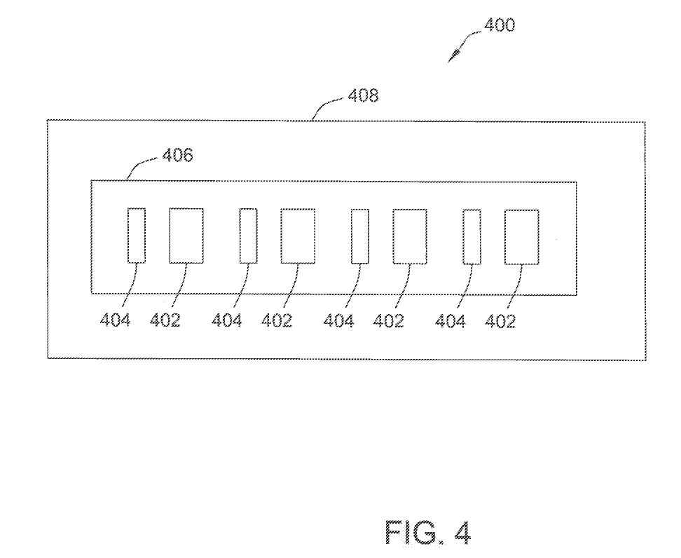

FIG. 4 is a schematic diagram of one embodiment of a Hall sensor 400 that may be used to implement each of first vertical Hall sensor 208 and second vertical Hall sensor 210. Sensor 400 includes a plurality of PSDs 402 and NSDs 404 in an NWELL 406. An additional PSD 408 functions as a guarding. Those of skill in the art will appreciate that other sensor architectures may be used to implement first and second vertical Hall sensors 208 and 210.

Referring back to FIG. 2, Hall sensors 206, 208, and 210 form a multi-dimensional high magnetic field sensor that, due to its well-defined sensitivity and magnet field linearity, provides advantages over other magnetic field sensors (e.g., giant magnetoresistance (GMR) sensors and reed switches). Specifically, the multi-dimensional sensor formed by combining Hall sensors 206, 208, and 210 has the capability of differentiating between magnetic fields generated by an MRI scanner and magnetic fields generated by a handheld magnet, as described herein. Further, using the multi-dimensional sensor, MRI scanner detection is independent of the physical orientation of AIMD 100, which allows automatic MRI mode module 200 to accurately and reliability detect the presence of an MRI scanner.

Specifically, horizontal Hall sensor 206 is configured to detect relatively smaller magnetic fields, such as those generated by handheld magnets. In contrast, first and second vertical Hall sensors 208 and 210 are configured to detect relatively larger magnetic fields, such as those generated by MRI scanners. For example, in some embodiments, horizontal Hall sensor 206 is capable of detecting magnetic fields greater than or equal to 10 Gauss (G), and first and second vertical Hall sensors 208 and 210 are capable of detecting magnetic fields greater than or equal to 100 G. Alternatively, hall sensors 206, 208, and 210 may be capable of detecting any magnetic field strength that enables AIMD 100 to function as described herein. First and second vertical hall sensors 208 and 210 detect magnetic fields along both x-axis 102 and y-axis 104 in both polarities. Accordingly, regardless of the physical orientation of AIMD 100, first and second vertical hall sensors 208 and 210 are able to detect the presence of an MRI scanner.

The bipolarity of Hall sensors 206, 208, and 210 allows a patient implanted with AIMD 100 to enter an MRI scanner head or feet first, as AIMD 100 is able to detect magnetic fields in both positive and negative polarities. Accordingly, the multi-dimensional functionality of Hall sensors 206, 208, and 210 allows automatic MRI mode module 200 to reliably detect the presence of an MRI scanner without requiring oversight (e.g., from a physician).

As noted above, automatic MRI mode module 200 is also capable of distinguishing between fields from an MRI scanner and fields from handheld magnets. The amplitude of magnetic fields experienced by Hall sensors 206, 208, 210 is proportional to output voltages of Hall sensors 206, 208, 210 provided to processor 202. This allows AIMD 100 to avoid entering the MRI mode when only a handheld magnet is present.

Once AIMD 100 automatically detects the presence of an MRI scanner using automatic MRI mode module 200, AIMD 100 initiates programming to place AIMD 100 in the MRI mode. In one embodiment, the MRI mode lasts for a predetermined amount of time. The predetermined amount of time may be relatively short (e.g., five minutes) or relatively long (e.g., two to four hours). In some embodiments, the physician may specify the predetermined amount of time. Once the predetermined amount of time expires (i.e., after the patient has left the MRI scanner), AIMD 100 automatically returns to its default programming (e.g., a physician-recommended pacing therapy). The predetermined amount of time may be tracked using, for example, a digital timer implemented using processor 202. Accordingly, the patient does not need to visit a physician before or after the MRI procedure to have the MRI mode selectively activated and deactivated.

In another embodiment, instead of waiting for a predetermined amount of time to expire, once the MRI mode is initiated, automatic MRI mode module 200 periodically (e.g., at a rate of 8 Hz) samples Hall sensors 206, 208, 210 to detect the MRI scanner. Once automatic MRI mode module 200 no longer detects the MRI scanner for a predetermined time period, AIMD 100 returns to its default programming. This decreases the amount of time that AIMD 100 is in the MRI mode. The predetermined time period may be relatively short (e.g., five minutes) or relatively long (e.g., two to four hours).

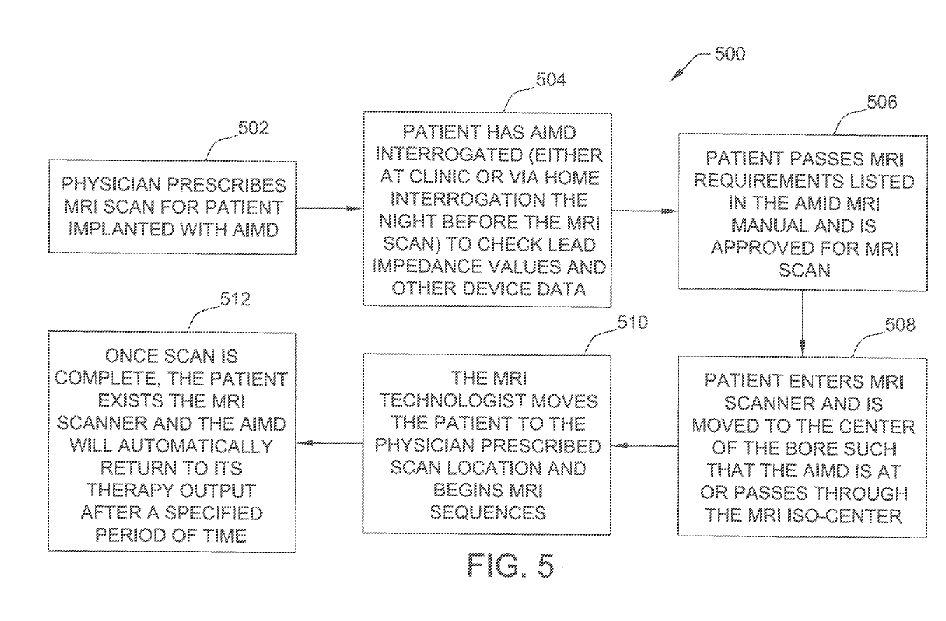

FIG. 5 is a flow diagram illustrating one embodiment of a process 500 of a patient implanted with AIMD 100 undergoing an MRI scan. At block 502, a physician prescribes an MRI scan for a patient implanted with AIMD 100. At block 504, AIMD 100 is interrogated to ensure AIMD 100 is operating properly. For example, lead impedance values and other device data may be verified.

At block 506, the patient is approved for the MRI scan. At block 508, the patient enters the MRI scanner and is moved to the center of a bore of the MRI scanner, such that AIMD 100 is located at or passes through an iso-center of the MRI scanner. This ensures that AIMD 100 detects the presence of the MRI scanner. At block 510, the patient is moved (e.g., by an MRI technologist) to a scan location prescribed by the physician, and the MRI scan begins. At block 512, after the MRI scan is complete, the patient exits the MRI, and AIMD 100 returns to its default programming.

In this embodiment, to detect ambient magnetic fields, processor 202 samples Hall sensors 206, 208, and 210 periodically (e.g., at a rate of approximately 8 Hz). Initially, to conserve energy, only horizontal Hall sensor 206 may be sampled. For example, if no handheld magnet or MRI scanner is present, horizontal Hall sensor 206 will not trigger, and automatic MRI mode module 200 returns to an idle state. However, if horizontal Hall sensor 206 is triggered, sampling of first and second vertical Hall sensors 208 and 210 is initiated. If only a handheld magnet is present, first and second vertical Hall sensors 208 and 210 will not trigger, and AIMD 100 will enter a magnet mode. However, if an MRI scanner is present, first and second vertical Hall sensors 208 and 210 will trigger, and AIMD 100 will enter the MRI mode. In other embodiments, all Hall sensors 206, 208, and 210 may be sampled simultaneously. However, this is relatively energy inefficient, and may reduce the lifespan of AIMD 100.

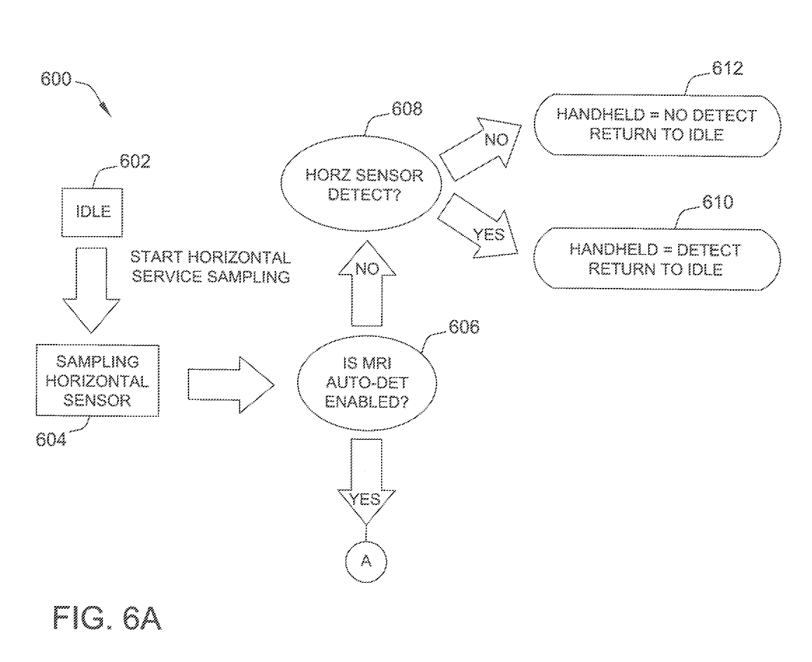

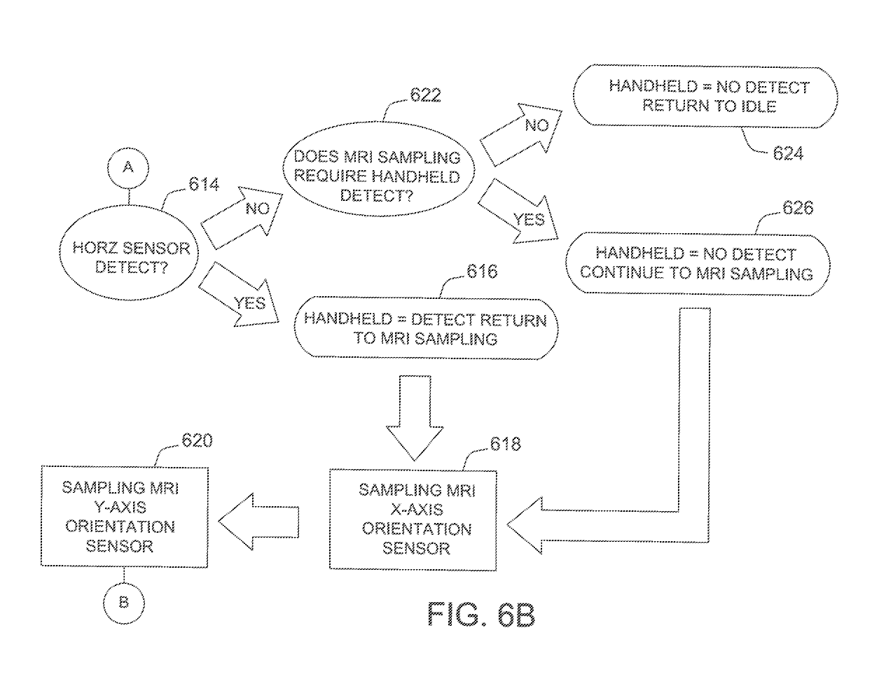

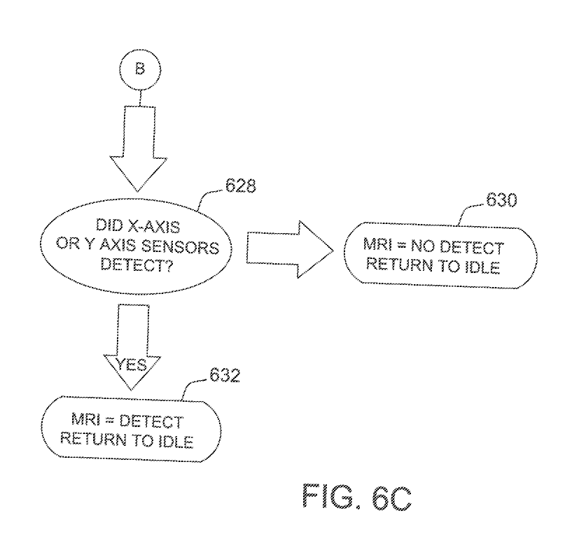

FIGS. 6A-6C are a flow diagram illustrating one embodiment of a method 600 for detecting an MRI scanner. Method 600 may be implemented, for example, using AIMD 100 having automatic MRI mode module 200 (shown in FIGS. 1 and 2, respectively).

Automatic MRI mode module 200 is initially in an idle state, as shown at block 602. At block 604, horizontal Hall sensor 206 is sampled. In some embodiments, the MRI auto-detection functionality may be selectively disabled. Accordingly, at block 606, it is determined whether the MRI auto-detection functionality is enabled. If the MRI auto-detection functionality is disabled, flow proceeds to block 608. At block 608, if horizontal Hall sensor 206 detects a magnetic field, flow proceeds to block 610, a magnet mode is initiated, and automatic MRI mode module 200 returns to the idle state. If, however, horizontal Hall sensor 206 does not detect a magnetic field, flow proceeds to block 612, and automatic MRI mode module 200 simply returns to the idle state (e.g., without initiating a magnet mode or MRI mode).

From block 606, if the MRI auto-detection functionality is disabled, flow proceeds to block 614. If horizontal Hall sensor 206 detects a magnetic field at block 614, flow proceeds to block 616, a magnet mode is initiated, and flow proceeds to block 618 to initiate sampling of first and second vertical Hall sensors 208 and 210. Specifically, at block 618, first vertical Hall sensor 208 aligned with x-axis 102 is sampled, and flow proceeds to block 620, wherein second vertical hall sensor 210 aligned with y-axis 104 is sampled.

If horizontal Hall sensor 206 does not detect a magnetic field at block 614, flow proceeds to block 622, wherein it is determined whether detecting a magnetic field using horizontal Hall sensor 206 is a prerequisite for detecting an MRI scanner. That is, in some embodiments, for redundancy, it may be desirable to sample first and second vertical hall sensors 208 and 210 even if horizontal Hall sensor 206 is not initially triggered.

If detecting a magnetic field using horizontal Hall sensor 206 is a prerequisite, flow proceeds to block 624, and automatic MRI mode module 200 simply returns to the idle state (e.g., without initiating a magnet mode or MRI mode). If detecting a magnetic field using horizontal Hall sensor 206 is not a prerequisite, flow proceeds to block 626, and then proceeds to blocks 618 and 620.

From block 620, flow proceeds to block 628, where it is determined whether at least one of first and second vertical Hall sensors 208 and 210 detected a magnetic field. If neither of first and second vertical Hall sensors 208 and 210 detected a magnetic field, flow proceeds to block 630, and automatic MRI mode module 200 simply returns to the idle state (e.g., without initiating an MRI mode). If, in contrast, at least one of first and second vertical Hall sensors 208 and 210 detected a magnetic field, flow proceeds to block 632, the MRI mode is initiated, and automatic MRI mode module 200 returns to the idle state.

To ensure proper detection of an MRI scanner by AIMD 100, AIMD 100 including automatic MRI mode module 200 may have certain labelling requirements. For example, labelling requirements may specify that an MRI technologist move AIMD 100 (and the patient) to the center of the bore of the MRI scanner before any MRI sequences are initiated. This will ensure that AIMD 100 detects MRI scanner and automatically enters the MRI mode.

In some embodiment, one or more secondary sensors (not shown) may be used to verify the detection capabilities of Hall sensors 206, 208. and 210. For example, a three-dimensional MEMS sensor, a telemetry coil, a gradient field detector, and/or a lead conductor sensing front end may be used as a secondary sensor. The secondary sensor may be enabled for a predetermined period of time when horizontal Hall sensor 206 is triggered, but first and second vertical Hall sensors 208 and 210 are not triggered. In such embodiments, the secondary sensor triggers initiation of the MRI mode if the secondary sensor detects the presence of a switching gradient field.

In the embodiments described herein, AIMD 100 may generate an alert when initiating the MRI mode and/or when exiting the MRI mode. The alert may include, for example, an audible alert or vibration of AIMD 100 that is detectable by the patient. Alternatively, AIMD 100 may generate any suitable alert.

The embodiments described herein provide systems and methods for automatically detecting the presence of an MRI scanner, and automatically initiating an MRI mode for an AIMD in response to the detection. The embodiments sample horizontal and vertical Hall sensors to accurately detect the presence of the MRI scanner.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An active implantable medical device (AIMD) comprising:
a processor;
a first magnetic field sensor communicatively coupled to the processor and configured to detect magnetic fields generated by a handheld magnet; and
at least one second magnetic field sensor communicatively coupled to the processor and configured to detect magnetic fields generated by a magnetic resonance imaging (MRI) scanner, wherein the processor is configured to:
sample the first magnetic field sensor and the at least one second magnetic field sensor to detect the presence of the MRI scanner; and
automatically initiate an MRI mode for the AIMD based on the detection.

2. The AIMD of claim 1, wherein the at least one second magnetic field sensor comprises two Hall sensors.

3. The AIMD of claim 2, wherein the AIMD defines three perpendicular axes, wherein the first magnetic field sensor is aligned with a first axis of the three perpendicular axes, wherein one of the two Hall sensors is aligned with a second axis of the three perpendicular axes, and wherein the other of the two Hall sensors is aligned with a third axis of the three perpendicular axes.

4. The AIMD of claim 1, wherein the AIMD is one of a pacemaker, a cardiac resynchronization therapy defibrillator (CRT-D), an insertable cardiac monitor (ICM), a deep brain stimulation (DBS) device, a dorsal root ganglia (DRG) stimulation device, a cardiac resynchronization therapy pacer (CRT-P), or a leadless cardiac pacemaker (LCP).

5. The AIMD of claim 1, wherein the processor is further configured to, after initiating the MRI mode, return to default programming after a predetermined amount of time expires.

6. The AIMD of claim 1, wherein the processor is further configured to:
continue to sample the first magnetic field sensor and the at least one second magnetic field sensor after initiating the MRI mode; and
return to default programming once the presence of the MRI scanner is no longer detected for a predetermined time period.

7. The AIMD of claim 1, wherein to sample the first magnetic field sensor and the at least one second magnetic field sensor, the processor is configured to:
sample only the first magnetic field sensor until a magnetic field is detected by the first magnetic field sensor;
initiate sampling of the at least one second magnetic field sensor when the magnetic field is detected by the first magnetic field sensor; and
detect the presence of the MRI scanner when the magnetic field is also detected by the at least one second magnetic field sensor.

8. The AIMD of claim 1, wherein the first magnetic field sensor and the at least one second magnetic field sensor are bipolar Hall sensors.

9. The AIMD of claim 1, wherein the processor is further configured to generate an alert when the MRI mode is initiated.

10. An automatic magnetic resonance imaging (MRI) mode module for use in an active implantable medical device (AIMD), the automatic MRI mode module comprising:
a processor;
a first magnetic field sensor communicatively coupled to the processor and configured to detect magnetic fields generated by a handheld magnet; and
at least one second magnetic field sensor communicatively coupled to the processor and configured to detect magnetic fields generated by a magnetic resonance imaging (MRI) scanner, wherein the processor is configured to:
sample the first magnetic field sensor and the at least one second magnetic field sensor to detect the presence of the MRI scanner; and
automatically initiate an MRI mode based on the detection.

11. The automatic MRI mode module of claim 10, wherein the at least one second magnetic field sensor comprises two Hall sensors.

12. The automatic MRI mode module of claim 10, wherein the processor is further configured to, after initiating the MRI mode, return to default programming after a predetermined amount of time expires.

13. The automatic MRI mode module of claim 10, wherein the processor is further configured to:
continue to sample the first magnetic field sensor and the at least one second magnetic field sensor after initiating the MRI mode; and
return to default programming once the presence of the MRI scanner is no longer detected for a predetermined time period.

14. The automatic MRI mode module of claim 10, wherein to sample the first magnetic field sensor and the at least one second magnetic field sensor, the processor is configured to:
sample only the first magnetic field sensor until a magnetic field is detected by the first magnetic field sensor;
initiate sampling of the at least one second magnetic field sensor when the magnetic field is detected by the first magnetic field sensor; and
detect the presence of the MRI scanner when the magnetic field is also detected by the at least one second magnetic field sensor.

15. The automatic MRI mode module of claim 10, further comprising at least one secondary sensor configured to verify detection capabilities of the first magnetic field sensor and the at least one second magnetic field sensor, wherein the at least one secondary sensor comprises one of a MEMS sensor, a telemetry coil, a lead conductor sensing front end, and a gradient field detector.

16. The automatic MRI mode module of claim 10, wherein the processor is further configured to generate an alert when the MRI mode is initiated.

17. A method for automatically initiating a magnetic resonance imaging (MRI) mode for an active implantable medical device (AIMD), the method comprising:
sampling, using a processor, a first magnetic field sensor and at least one second magnetic field sensor, wherein the first magnetic field sensor is configured to detect magnetic fields generated by a handheld magnet, and wherein the at least one second magnetic field sensor is configured to detect magnetic fields generated by an MRI scanner;
detecting the presence of the MRI scanner based on the sampling; and
automatically initiating the MRI mode based on the detection.

18. The method of claim 17, wherein sampling a first magnetic field sensor and at least one second magnetic field sensor comprises sampling the first magnetic field sensor and two second magnetic field sensors.

19. The method of claim 17, further comprising returning to default programming from the MRI mode after a predetermined amount of time expires.

20. The method of claim 17, further comprising:
continuing to sample the first magnetic field sensor and the at least one second magnetic field sensor after initiating the MRI mode; and
returning to default programming once the presence of the MRI scanner is no longer detected for a predetermined time period.
